(19) United States
(12) Patent Application Publication
(10) Pub. No.: US 2007/0026388 A1
(43) Pub. Date: Feb. 1, 2007

(54) Analyte detection using carbon nanotubes

(76) Inventors: Stephen K. Doorn, Los Alamos, NM (US); Michael J. O'Connell, San Jose, CA (US); Hsing-Lin Wang, Los Alamos, CA (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(21) Appl. No.: 11/192,203

(22) Filed: Jul. 28, 2005

(51) Int. Cl. C12Q 1/70; C12Q 1/68; G01N 33/53

(52) U.S. Cl. 435/5; 435/6; 435/7.1; 977/900; 977/924

(57) ABSTRACT

A method for detecting analytes involves measuring the fluorescence of a sample of fluorescence-attenuated carbon nanotubes, then exposing the sample to a target analyte, and then measuring the fluorescence again. The fluorescence-attenuated nanotubes include a chemical having quenching portion that interacts with the nanotubes and attenuates the fluorescence, a receptor portion that binds to a target analyte and a tether portion that connects the receptor portion to the quenching portion. If the target analyte binds to the receptor portion of the chemical, the interaction between the chemical and the nanotubes and the chemical is weakened and the original fluorescence of the carbon nanotubes is restored.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates generally to analyte detection and more particularly to a method for analyte detection using carbon nanotubes.

### BACKGROUND OF THE INVENTION

Methods and systems for detecting and measuring trace amounts of analytes such as proteins, microorganisms, pharmaceuticals, viruses, antibodies, and nucleic acids with high selectivity are of great value to researchers and clinicians. These methods and systems are often based on molecular recognition. Molecular recognition of a protein, for example, depends largely on the exposed surface amino acid residues of the polypeptide chain. These residues can form weak noncovalent bonds with other molecules. An effective binding between the protein and some other species generally requires that many weak bonds form simultaneously between the protein and that species. These bonds form at the “binding site” of the protein. The binding site is usually a cavity in the protein that is formed by a specific arrangement of amino acids that often belong to widely separated regions of the polypeptide chain and represent only a minor fraction of the total number of amino acids present in the chain. The species, typically a molecule or ion, must fit precisely into the binding site for effective binding to occur. The shape of these binding sites can differ greatly among different proteins, and even among different conformations of the same protein. Even slightly different conformations of the same protein may differ greatly in their binding abilities (see, for example: Alberts et al., “Molecular Biology of the Cell”, 2^{nd} edition, Garland Publishing, Inc., New York, 1989; and Lodish et al., “Molecular Cell Biology”, 4^{th} edition, W. H. Freeman and Company, 2000).

The high degree of specificity involved in molecular recognition has led to the development of assay methods and systems, such as the enzyme-linked immunosorbant assay (ELISA), which is widely used for identifying the presence and biological activity of a wide range of proteins, antibodies, cells, viruses, and the like. ELISA is a multi-step assay in which an analyte biomolecule is first bound to an antibody tethered to a surface. A second antibody then binds to the biomolecule. In some cases, the second biomolecule antibody is biotinylated to bind a third protein (e.g. avidin or strepavidin). This protein is tethered either to an enzyme, which creates a chemical cascade for an amplified calorimetric change, or to a fluorophore, for fluorescent tagging.

Analyte detection sometimes relies upon binding events that essentially turn on and off a property such as fluorescence. An example of this type of detection can be found in “Highly Sensitive Biological and Chemical Sensors Based on Reversible Fluorescence Quenching in a Conjugated Polymer” by Chen et al., Proc. Natl. Acad. Sci., vol. 96 (1999) pp.12287-12292, incorporated by reference herein. Chen et al. use a fluorescent polymer and a quencher molecule that quenches the fluorescence of the polymer by forming a charge transfer complex with the polymer. The quencher molecule is also capable of binding to one or more target analytes. In the presence of those target analytes, the quencher molecule of the charge transfer complex binds to the target analyte, which results in dissociation of the quencher molecule from the polymer and restoration of the fluorescence of the polymer. This type of detection can also be found in “Conjugated Polymers as Fluorescence Quenchers and Their Applications for Bioassay”, Song et al., Chem. Mater., vol. 14 (2002) pp. 2342-2347; and in U. S. Pat. No. 6,589,731 entitled “A Method for Detecting Biological and Chemical Agents”, Chen et al., issued Jul. 08, 2003, all incorporated by reference.

Methods for sensitive biomolecular and biochemical analyte detection remain desirable.

Accordingly, an object of the present invention is to provide a sensitive method for detecting biological and biochemical analytes.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

In accordance with the purposes of the present invention, as embodied and broadly described herein, the present invention includes method for detecting a target analyte. The method involves measuring a property of a sample of property-attenuated carbon nanotubes wherein the property is selected from the group consisting of fluorescence and conductance, thereafter exposing the sample to a target analyte, and thereafter measuring the property again.

The invention also includes a method for detecting a target analyte. The method involves measuring a property selected from the group consisting of fluorescence and electrical conductivity from a sample of carbon nanotubes; thereafter exposing the sample of carbon nanotubes to a chemical that attenuates said property; thereafter measuring said property again; thereafter exposing the sample of carbon nanotubes to a target analyte; and thereafter measuring the property again.

### BRIEF DESCRIPTION OF THE DRAWINGS

The accompanying drawings, which are incorporated in and form a part of the specification, illustrate the embodiment(s) of the present invention and, together with the description, serve to explain the principles of the invention. In the drawings:

FIG. 1 is a schematic representation of an embodiment of the present invention involving analyte detection using changes in fluorescence from carbon nanotubes.

FIG. 2 shows fluorescence spectra of a suspension of single walled carbon nanotubes before and after the addition of 4-amino-1,1′-azobenzene-3,4′-disulfonic acid (AB).

FIG. 3 shows the visible-near infrared (VIS-NIR) absorbance spectrum of the carbon nanotubes after addition of AB.

FIG. 4 shows the structure of biotinylated Lucifer yellow, biotinylated anthracene, and biotinylated azobenzene, which were used to demonstrate the method of the present invention.

FIG. 5 shows an absorbance spectrum of a suspension of carbon nanotubes, another spectrum after the addition of biotinylated Lucifer yellow to the suspension, and several spectra after subsequent additions of aliquots of the target analyte avidin to the suspension; and

FIG. 6a shows a fluorescence spectrum of carbon nanotubes and a spectrum of the carbon nanotubes after the addition of biotinylated anthracene, and FIG. 6b shows six fluorescence spectra that demonstrate fluorescence recovery after subsequent aliquot additions of avidin.

### DETAILED DESCRIPTION

The present invention is concerned with analyte detection using carbon nanotubes. According to one embodiment of the invention, the fluorescence of an aqueous suspension of carbon nanotubes is measured, and then the suspension of carbon nanotubes is combined with a chemical that attenuates the fluorescence of the carbon nanotubes. The chemical has a quenching portion that interacts with the carbon nanotubes and attenuates their fluorescence by, for example, accepting electrons from the carbon nanotubes. The chemical also has a receptor portion capable of binding to a target analyte and a tethering portion that connects the quenching portion to the receptor portion. In the presence of a target analyte that can bind to the receptor portion of the chemical, the receptor portion binds to the target analyte, resulting in lessening the interaction of the quenching portion with the carbon nanotubes to the extent that the fluorescence of the carbon nanotubes is restored.

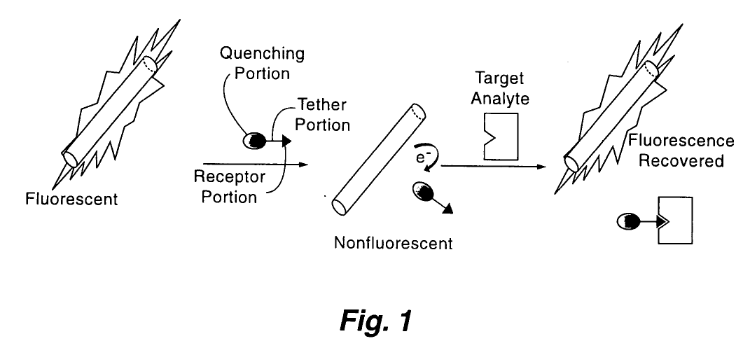

FIG. 1 schematically illustrates the method of the present invention. Going from left to right, a fluorescent carbon nanotube is exposed to a chemical that interacts with the carbon nanotube and attenuates its fluorescence. In the presence of target analyte, the receptor portion of the chemical binds to a target analyte, which results in dissociation of the chemical from the carbon nanotube and restoration of the fluorescence of the carbon nanotube.

Chemicals with a quenching portion, a receptor portion, and a tether portion have been described by, for example, Chen et al. in U. S. Pat. No. 6,589,731, and by David G. Whitten et al. in U. S. Pat. No. 6,743,640 entitled “Fluorescent Polymer-QTL Approach to Biosensing,” which issued on Jun. 1, 2004, both incorporated by reference herein.

The receptor portion of the chemical used with this invention is capable of molecular recognition with specific analytes, preferably biological or biochemical analytes. Preferred receptor portions include, but are not limited to, chemical ligands, antibodies, polynucleotides, antigens, polypeptides, and polysaccharides. Combinations of pairs that are categorizable as receptor-analyte pairs are well know to those skilled in the art. Some of these pairs include hormone-hormone receptor pairs, polynucleotide strand-complementary polynucleotide strand pairs, enzyme-enzyme cofactor or inhibitor pairs, avidin-biotin, protein A-immunoglobulin, lectin-specific carbohydrate, and cholera toxin (CT) and ganglioside GM1.

The tethering portion that connects the quenching portion to the receptor portion is of a length adapted to allow for the receptor portion to extend to or reach the part of the target analyte that binds to the receptor portion. The tethering portion can be tailored to the necessary length to allow the receptor portion to reach the binding site on a target analyte, preferably a biological or biochemical analyte. This length can be as short as a single linking atom or may be up to as many as about 100 atoms in length, preferably from about 3 to about 25 atoms in length for the tethering portion. Often, the receptor portion and the target analyte being detected and/or measured are specific for one another. This specificity can be of a chemical nature, of a geometric nature, or both. The recognition can be as specific as a “lock and key” arrangement where only a single receptor will function to join with the analyte.

The chemical need not completely reduce the fluorescence from the carbon nanotubes, but instead form a complex with the carbon nanotubes having an attenuated signal that could be distinguishable from the signal resulting from a subsequent binding event. In a preferred embodiment of the present invention, the reduced fluorescence signal of the complex may provide a low background or baseline property measurement. Then, upon dissociation of the complex, a fluorescence signal from the carbon nanotubes is easily detectable. Also, the formation of the complex could also lead to a spectral shifting, such as a shifting of a fluorescence wavelength.

The chemical that forms a complex with the carbon nanotubes is adapted for separation from carbon nanotubes upon the binding of the receptor portion of the chemical to a target analyte. The affinity or binding constant between the receptor portion and the target analyte is high enough for separation of the chemical from the carbon nanotubes, allowing for the detection of the change in fluorescence. Thus, complex formation leads to a reduced fluorescence signal from the carbon nanotubes, and the removal of the bound chemical results in the regeneration of the fluorescent event and allows for fluorescence detection.

Target analytes recognizable by the receptor portion include, for example, a biomolecule for which a ligand exists or may be synthesized.

The detectable changes in fluorescence that accompany binding of target analytes to the receptor portion of the chemical can be measured using, for example, a fluorescence spectrophotometer. A current/voltage meter can be used to measure changes in conductance.

The carbon nanotubes used for demonstration purposes with the present invention were supplied by RICE UNIVERSITY. These carbon nanotubes were prepared using the known “High Pressure CO Process” (i.e. the HiPco Process), which has been described by Nikolaev et al. in “Gas Phase Catalytic Growth of Single-Walled Carbon Nanotubes From Carbon Monoxide,” Chem. Phys. Lett. vol. 313 (1999) pp. 91-97, incorporated by reference herein.

Surfactants for preparing suspensions of carbon nanotubes used with the invention include, but are not limited to, TRITON X-405, poly(ethylene glycol)-co-poly(propylene glycol), sodium cholate, sodium dodecyl benzene sulfonate (SDDBS), sodium dodecyl sulfate (SDS), and the like (see: O'Connell et al. “Band Gap Fluorescence From Individual Single-Walled Carbon Nanotubes, ” Science (2002) vol. 297 pp. 593-596, incorporated by reference herein). SDS is a preferred surfactant.

Other materials can also be used for preparing aqueous suspensions of carbon nanotubes. Water-soluble polymers, for example, may be used. The polymers may be cationic, anionic, or neutral. Examples include, but are not limited to, poly vinyl pyrrolidone (PVP), poly(allylamine hydrochloride) PAH, poly(ethylenimine) (PEI), poly(diallyl- dimethylammonium chloride) (PDDA), polystyrene sulfonate (PSS), polyacrylic acid (PAA), and biopolymers including polynucleic acids and polypeptides.

The method of the invention is now described by way of example. 7,7,8,8-tetracyanoquinodimethane (TCNQ), 2,3,5,6-tetrafluoro-7,7,8,8-tetracyanoquinodimethane (TFTCNQ), mordant yellow 10 (MY), and 4-amino-1,1′-azobenzene-3,4′-disulfonic acid (AB) were purchased from ALDRICH and used without further purification. Spectral changes induced by reactions of carbon nanotubes were monitored by fluorescence and absorption spectroscopy. The spectra were recorded for samples in stirred, quartz cuvettes capped with septa. Reagents were added to the cuvette using a syringe. Absorbance measurements were made with fiber optic spectrometers (STELLAR NET) covering a range from about 190 nm to about 1700 nm. Fluorescence measurements with a 785 nm diode laser excitation source were made with a FT-Raman instrument (BRUKER RFS 100/S) modified for near-infrared (NIR) emission. Data was collected at 8 cm⁻¹ resolution and averaged over 16 scans for spectra collected at 15 second intervals, or over 64 scans for spectra collected at 2 minute intervals.

### EXAMPLE 1

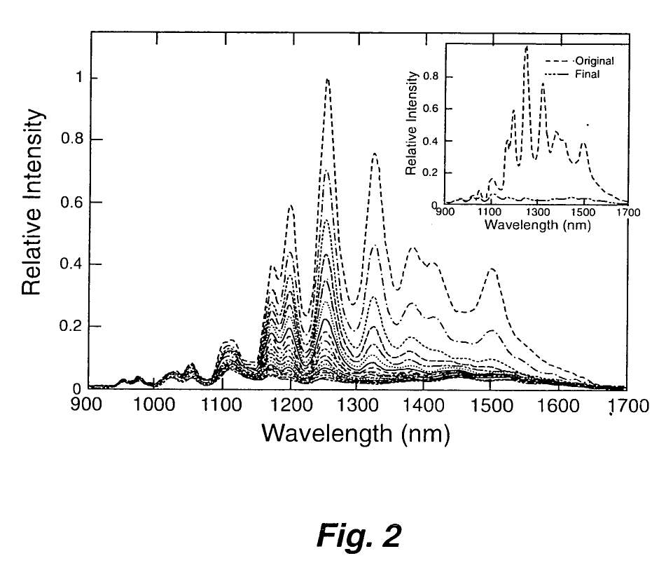

Fluorescence attenuation of carbon nanotubes. A sample of carbon nanotubes was suspended in an aqueous solution of sodium dodecyl sulfate (SDS, 1 percent). Addition of 4-amino-1,1′-azobenzene-3,4′-disulfonic acid (AB) to the suspension resulted in a rapid attenuation of the nanotube fluorescence intensity. FIG. 2 shows plots of fluorescence spectra taken at intervals over about 5 minutes. The spectra show that the loss in fluorescence intensity starts with the larger diameter, lower energy bandgap nanotubes and progresses to the smaller diameter, higher energy bandgap nanotubes at a given concentration. Similar behavior was reported by Zheng et al. in “Structure-Based Carbon Nanotube Sorting By Sequence-Dependent DNA Assembly, ” Science, vol. 302 (2003) pp. 1545-1548, incorporated by reference herein. According to Zheng et al., after adding an oxidizing agent to a sample of DNA-wrapped nanotubes, the absorbance of the nanotubes was bleached, and the nanotubes having smaller bandgap are affected the sooner than those having a larger bandgap.

The inset of FIG. 2 shows a fluorescence spectrum taken 5 minutes after 1 μmol of AB was added to a carbon nanotube suspension. The fluorescence intensity for nearly all the carbon nanotubes was attenuated. Addition of more AB resulted in a complete loss of nanotube fluorescence.

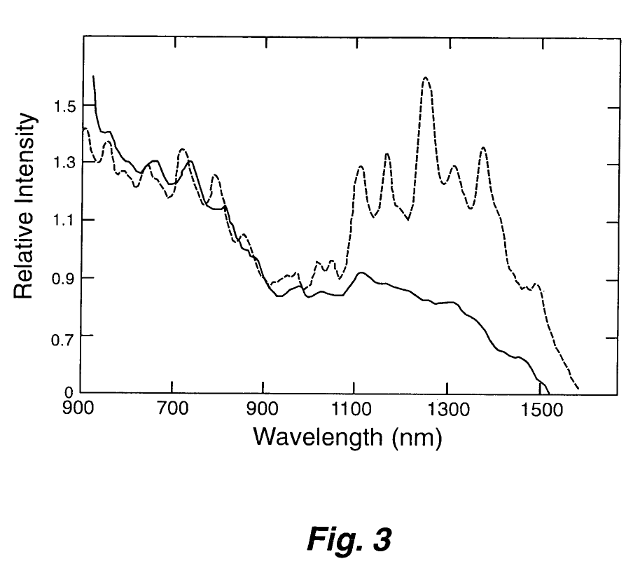

FIG. 3 shows the visible-near infrared (VIS-NIR) absorbance spectrum of the carbon nanotubes after addition of AB. As FIG. 3 shows, only the first van Hove transitions (greater than 900 nm) become bleached. The second van Hove absorbance peaks in the 500 to 900 nm region remained, which is consistent with an electron-transfer reaction occurring from the top of the valence band. The second van Hove absorbance peaks, however, were slightly red shifted. The red shift is believed to result from disruption of the SDS micelle structure and/or the noncovalent interaction of the charge transfer molecule with the nanotube sidewall. The shift therefore is consistent with formation of a charge transfer complex. Similar red shifting has been observed when the nanotube SDS environment is exchanged for a polymer [see: M. J. O'Connell et al., “Band Gap Fluorescence From Individual Single-Walled Carbon Nanotubes, ” Science, vol. 297 (2002) pp. 593-596] or for other surfactants [see: V. C. Moore et al., “Individually Suspended Single-Walled Carbon Nanotubes in Various Surfactants, ” NanoLeft., vol. 3 (2003) pp. 1379-1382]. These observations demonstrate the extreme sensitivity of nanotubes to their environment.

Reaction of carbon nanotubes with the other organic acceptor molecules such as TCNQ, TFTCNQ, and MY resulted in similar spectral behavior, but with differing bleaching kinetics and effectiveness.

### EXAMPLE 2

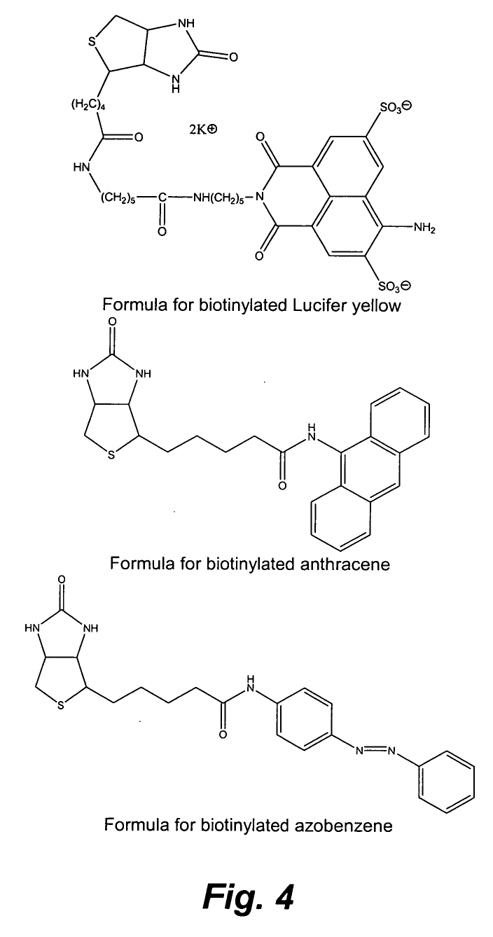

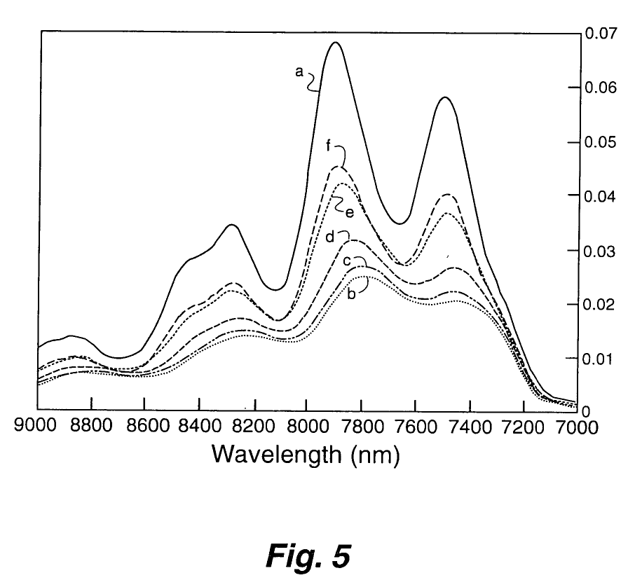

Fluorescence attenuation using biotinylated Lucifer yellow, and fluorescence recovery using avidin. A sample of carbon nanotubes was suspended in an aqueous solution of sodium dodecyl sulfate (SDS, 10 milligrams per liter). The fluorescence signal from the carbon nanotubes was measured (see FIG. 5, spectrum (a)). After measuring the fluorescence, biotinylated Lucifer yellow (10 μL of 0.1 molar solution), whose chemical formula is shown in FIG. 4, was added to the suspension and the fluorescence signal from the carbon nanotubes was measured again (FIG. 5, spectrum (b)).

To the resulting suspension were added 10 μL to 200 μL aliquots of a 10⁻⁵ molar solution of the target analyte avidin. The fluorescence spectra were recorded after each addition. Spectrum (c) was taken after 10 μL was added. Spectrum (d) was taken after a total of 50 μL was added. Spectrum (e) was taken after a total of 100 μL was added, and spectrum (f) was taken after a total of 200 μL was added. As FIG. 5 shows, the fluorescence was found to recover incrementally as each aliquot of avidin was added. The sensitivity to the target analyte for this EXAMPLE was low as about 10 nanomolar (nM).

### EXAMPLE 3

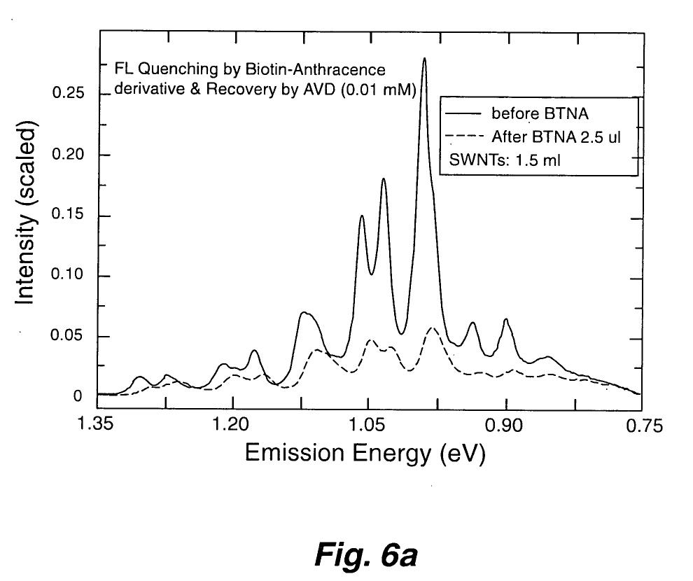

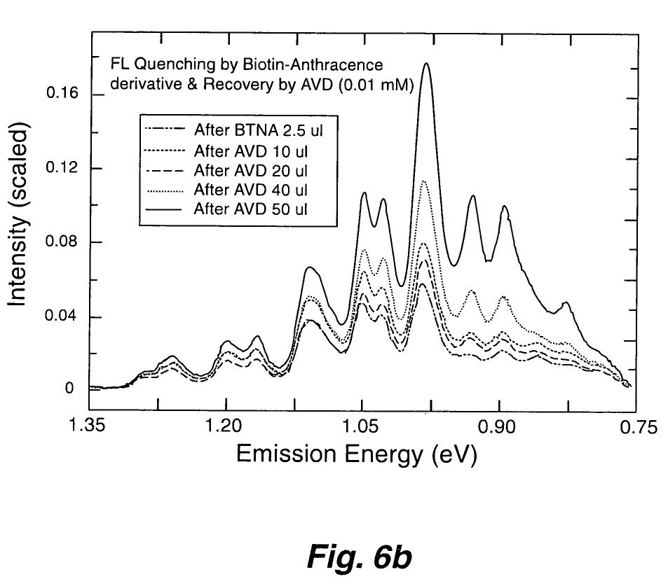

Fluorescence attenuation using biotinylated anthracene, and fluorescence recovery using avidin. A sample of carbon nanotubes was suspended in an aqueous solution of sodium dodecyl sulfate (SDS, 10 milligrams per liter). The fluorescence signal from the carbon nanotubes was measured (see FIG. 6a).

After measuring the fluorescence, biotinylated anthracene (BTNA) (10 μL of 0.1 molar solution), whose chemical formula is shown in FIG. 4, was added to the suspension and the fluorescence signal from the carbon nanotubes was measured again (see FIG. 6a). As FIG. 6a shows, the fluorescence spectrum after addition shows an attenuated fluorescence from the carbon nanotubes.

To the resulting suspension were added 10 to 60 μL aliquots of a 10⁻⁵ molar solution of avidin (AVD). Fluorescence spectra were recorded after each addition, and are shown in FIG. 6b. As FIG. 6b shows, the fluorescence signal after addition of avidin was found to recover incrementally as each aliquot of avidin was added. The sensitivity to the target analyte for this EXAMPLE was as low as about 10 nM.

### EXAMPLE 4

Fluorescence attenuation using biotinylated azobenzene, and fluorescence recovery using avidin. A sample of carbon nanotubes was suspended in an aqueous solution of sodium dodecyl sulfate (SDS, 10 milligrams per liter). The fluorescence signal from the carbon nanotubes was measured. After measuring the fluorescence, biotinylated azobenzene (10 μL of 0.1 molar solution), whose chemical formula is shown in FIG. 4, was added to the suspension and the fluorescence signal from the carbon nanotubes was measured again.

To the resulting suspension were added 10 μL to 200 μL aliquots of a 10⁻⁵ molar solution of the target analyte avidin. The fluorescence signals were found to recover incrementally as each aliquot of avidin was added.

### EXAMPLE 5

Conductance attenuation and recovery. In addition to a method for detecting analytes using changes in the fluorescence of carbon nanotubes, the invention also includes an analyte detection method using changes in conductance (i.e. electrical conductivity) of carbon nanotubes. An embodiment for conductivity-based detection may involve preparation of an assembly of carbon nanotubes on a substrate. Such an assembly could be prepared by, for example, depositing carbon nanotubes on a silicon substrate using a well-known technique such as chemical vapor deposition (CVD).

After depositing the nanotubes, electrodes would be deposited at different positions along the length of the nanotubes. Electrode deposition could be accomplished using any of a number of well-known nanolithography techniques. An embodiment may involve the deposition of two electrodes that are separated by a chosen distance along a nanotube.

After depositing the electrodes, the conductance of the nanotube segment located between the two electrodes would be measured. The nanotube segment in between the two electrodes would then be exposed to a solution of a chemical having a quenching portion, a receptor portion, and a tethering portion as described earlier. The chemical could be, for example, an organic chemical with a biotin receptor portion tethered to a quenching portion selected from azobenzenes, quinones, or similar species that can interact with carbon nanotubes as acceptors of electrons from the carbon nanotubes.

After exposure of the nanotube segment to the chemical, the conductivity of the nanotube segment would be measured again.

The assembly would be then exposed to a target analyte (biotin, for example) while monitoring nanotube conductance. Analyte binding to the receptor segment of the chemical would dissociate the chemical from the nanotube and result in recovery of the original measured conductance. Thus, recovery of the conductance indicates binding of the analyte to the chemical, and therefore detection of the analyte.

In summary, the invention includes a method for detecting analytes using chemicals that attenuate the fluorescence signal of carbon nanotubes and bind to target analytes. An exemplary chemical used for demonstrating fluorescence attenuation is Lucifer yellow tethered to the receptor biotin. This chemical reduces the nanotube fluorescence and also shifts the nanotube electronic spectrum. Addition of the target analyte avidin reverses the spectral shift and restores the nanotube fluorescence. Sensitivity has been demonstrated at the nanomolar (nM) level.

The foregoing description of the invention has been presented for purposes of illustration and description and is not intended to be exhaustive or to limit the invention to the precise form disclosed, and obviously many modifications and variations are possible in light of the above teaching. For example, the presence of oxygen may also have an effect on the charge transfer. Spectral bleaching at low pH was found to occur only in the presence of oxygen, and may be reversed by removing 0₂.

The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A method for detecting a target analyte comprising measuring a property of a sample of property-attenuated carbon nanotubes wherein the property is selected from the group consisting of fluorescence and conductance, thereafter exposing the sample to a target analyte, and thereafter measuring the property again.

2. The method of claim 1, wherein the sample of property-attenuated carbon nanotubes comprises an aqueous suspension.

3. The method of claim 1, wherein the sample of property-attenuated carbon nanotubes comprises a surfactant.

4. The method of claim 1, further comprising the step of forming property-attenuated carbon nanotubes by exposing carbon nanotubes to a chemical that comprises a quenching portion that attenuates the property of the carbon nanotubes, a receptor portion that binds to the target analyte, and a tether portion that connects the receptor portion to the quenching portion.

5. The method of claim 4, further comprising the step of measuring the property of the carbon nanotubes prior to forming the property-attenuated carbon nanotubes.

6. The method of claim 1, wherein the target analyte is selected from the group consisting of proteins, viruses, bacteria, cells, microorganisms, antibodies, nucleic acids, and toxins.

7. The method of claim 4, wherein the receptor portion of the chemical is selected from the group consisting of chemical ligands, antibodies, antibody fragments, oligonucleotides, antigens, polypeptides, glycolipids, proteins, enzymes, peptides, nucleic acids, and polysaccharides.

8. The method of claim 4, wherein the property-attenuated carbon nanotubes comprise a complex between carbon nanotubes and the chemical.

9. The method of claim 5, wherein exposure of the complex to the target analyte results in dissociation of the complex.

10. The method of claim 1, further comprising the step of forming carbon nanotubes by chemical vapor deposition of carbon nanotubes on a substrate.

11. The method of claim 10, further comprising the step of forming at least two electrodes on at least one of the carbon nanotubes.

12. A method for detecting a target analyte comprising:
measuring a property selected from the group consisting of fluorescence and conductance from a sample of carbon nanotubes;
thereafter exposing the sample of carbon nanotubes to a chemical that attenuates said property;
thereafter measuring said property again;
thereafter exposing the sample of carbon nanotubes to a target analyte; and thereafter
measuring the property again.

13. The method of claim 12, wherein the sample of carbon nanotubes comprises an aqueous suspension of carbon nanotubes.

14. The method of claim 12, wherein the aqueous suspension comprises a surfactant.

15. The method of claim 12, wherein the chemical that attenuates said property comprises a quenching portion that attenuates the fluorescence of the carbon nanotubes, a receptor portion that binds to the target analyte, and a tether portion that connects the receptor portion to the quenching portion.

16. The method of claim 15, wherein the receptor portion is selected from the group consisting of chemical ligands, antibodies, antibody fragments, oligonucleotides, antigens, polypeptides, glycolipids, proteins, enzymes, peptides, nucleic acids, and polysaccharides.

17. The method of claim 12, wherein the target analyte is selected from the group consisting of proteins, viruses, bacteria, cells, microorganisms, antibodies, nucleic acids, and toxins.

18. The method of claim 12, further comprising preparing the sample by depositing the carbon nanotubes on a substrate by chemical vapor deposition.

19. The method of claim 18, further comprising depositing two electrodes on at least one of the deposited carbon nanotubes.

20. The method of claim 19, further comprising measuring the conductance in between the two electrodes before exposing the carbon nanotube to the chemical.
